SEQUENCE LISTING (7 sequences)

SEQ ID NO: 1 - DNA, 47 bp, Artificial Sequence
  primer
ttcactgcagatgcataaggaagcatatgagtattcaacatttccgt

SEQ ID NO: 2 - DNA, 39 bp, Artificial Sequence
  primer
caaactgcagaagcttaccaatgcttaatcagtgaggca

SEQ ID NO: 3 - DNA, 33 bp, Artificial Sequence
  primer
caaggaattccatgggccatattcaacgggaaa

SEQ ID NO: 4 - DNA, 42 bp, Artificial Sequence
  primer
ccaggatcctttaaaaaaactcatcgagcatcaaatgaaact

SEQ ID NO: 5 - DNA, 21 bp, Artificial Sequence
  primer
ctatcgagcacagcgccaact

SEQ ID NO: 6 - DNA, 31 bp, Artificial Sequence
  primer
ccgaattcataaggaagcatatgagtattca

SEQ ID NO: 7 - DNA, 30 bp, Artificial Sequence
  primer
caacttttacgctggtgctatacaacgact

(19) United States
(12) Patent Application Publication
(10) Pub. No.: US 2006/0257976 A1
(43) Pub. Date: Nov. 16, 2006

(54) Methods and kits for propagating and evolving nucleic acids and proteins

(75) Inventors: Eugene Makeyev, Cambridge, MA (US); Dennis Bamford, Helsinki (FI)

Correspondence Address: FOLEY AND LARDNER LLP;SUITE 500, 3000 K STREET NW, WASHINGTON, DC, 20007, US

(73) Assignee: RNA-Line Oy

(21) Appl. No.: 10/559,575

(22) PCT Filed: Jun. 7, 2004
(86) PCT No.: PCT/FI04/00346

§ 371 Date: Apr. 18, 2006

(30) Foreign Application Priority Data
Jun. 6, 2003 (FI) 20030854

(51) Int. Cl. C12P 19/34; C12N 1/21

(52) U.S. Cl. 435/91.2; 435/325; 435/253.4

(57) ABSTRACT

A novel strategy for directed evolution of nucleic acids and proteins is described, in which target nucleic acid is copied by a polymerase devoid of proofreading function. Advantageous mutations generated during this process are recovered using an appropriate selection or screening procedure. The invention provides fast, inexpensive and non-laborious methods for practicing said strategy, which are utilized either separately or in combination with other methods for engineering biopolymers with desired properties. The invention furthermore provides kits for directed evolution according to the described methodology. In an aspect, the invention discloses methods and kits for producing nucleic acids encoding proteins with desired properties.

### FIELD OF THE INVENTION

The invention relates to the field of directed molecular evolution. More specifically, the invention relates to the use of the erroneous nature of RNA-based biological entity, in particular RNA virus replication for engineering nucleic acids and proteins with advantageous properties.

### BACKGROUND OF THE INVENTION

Proteins and nucleic acids are essential for the functioning of all biological systems. On the other hand, many proteins are of considerable importance for industry, medicine, agriculture, bioremediation, and other applications. Potential utility of nucleic acid-based enzymes, such as ribozymes, and binding molecules have also been discussed (Burgstaller et al., 2002; Cobaleda and Sanchez-Garcia, 2001; de Feyter and Li, 2000; Pohorille and Deamer, 2002; Robertson and Ellington, 2001; White et al., 2001). Practical applications often require properties that are irrelevant or even harmful for living organisms. As a consequence, the use of natural enzymes in industry can be limited by inefficient catalysis of non-natural substrates, low stability, low tolerance for changes in operating parameters, poor activity in non-aqueous solutions, or requirements of expensive cofactors (Farinas et al., 2001; Petrounia and Arnold, 2000). Similarly, antibodies obtained from immunized animals may not be adequate for diagnostic and therapeutic purposes due to low affinity, cross-reactivity, immuno-incompatibility, and other problems (Carter, 2001; Hudson and Souriau, 2003; Winter and Harris, 1993).

Two major strategies have been employed to improve protein performance: rational design and directed evolution (Arnold, 2001; Bornscheuer and Pohl, 2001). The first strategy can only be applied to proteins with known three-dimensional structures and remains challenging for practical use (Altamirano et al., 2000; Nixon et al., 1999; Quemeneur et al., 1998). On the contrary, directed evolution has become a popular approach to protein engineering and, furthermore, has been employed for selecting nucleic acid molecules with various biological activities (Farinas et al., 2001; Petrounia and Arnold, 2000).

All directed evolution protocols rely on a simple Darwinian optimization algorithm comprising the steps of diversification and selection. First, diversity is created within the population of target molecules. This is followed by selection that reveals improved variants that can be used as such or subjected to further rounds of evolution. Two distinct selection procedures have been used, bona fide selection and screening (see e.g. (Soumillion and Fastrez, 2001). Bona fide selection is based on survival or better propagation of the fittest variants of target molecules under selective conditions, which is conceptually similar to natural selection as understood in the theory of evolution. For the sake of simplicity, bona fide selection will be referred hereafter as “selection”. Term “screening” refers to manual or automated picking of preferred variants from a population of target molecules. This procedure can be likened to the artificial selection in Darwin's theory.

In the case of iterative diversification-selection rounds, the population of target molecules has to be occasionally replenished. Short peptides and oligonucleotides with predetermined sequence can be multiplied using chemical synthesis. In a more general case, RNA or DNA molecules are reproduced in vivo or in vitro through the template-copy mechanism according to the base complementarity rules. Proteins are commonly produced by translation of RNA templates (mRNAs) in either living cells (e.g. in phage, LacI, and cell-surface displays) (Chen and Georgiou, 2002; O'Neil and Hoess, 1995; Rader and Barbas, 1997; Schatz et al., 1996; Wittrup, 2001), or cell-free extracts (e.g. in mRNA display, different versions of ribosome display, and sorting in man-made compartments) (Amstutz et al., 2001; Tawfik and Griffiths, 1998). To ensure an adequate selection, proteins having desired properties (phenotype) have to be linked with the cognate nucleic acids (genotype).

A specialist in the field of directed evolution would recognize two major challenges in the relevant art. First, sufficiently large libraries of target molecules have to be constructed and searched for advantageous variants. Second, numerous directed evolution techniques allow for selecting improved binding activities, whereas only limited number of protocols can be used to alter enzymatic properties of target molecules.

With regard to the first challenge, diversification of target molecules is usually achieved using mutagenesis and/or recombination. Error-prone PCR and synthetic oligonucleotide-based techniques, such as e.g. cassette mutagenesis, have been methods of choice for diversifying nucleic acid populations in vitro (Trower, 1996). Similarly, in vitro recombination procedures have been described, including gene shuffling, exon shuffling, and nonhomologous random recombination (Bittker et al., 2002a; Coco et al., 2001; Kolkman and Stemmer, 2001; Kurtzman et al., 2001; Stemmer, 1994a; Stemmer, 1994b).

Because of the heavy use of PCR, DNA fragmentation, gel purification, DNA ligation, and other in vitro techniques, most of the above methods require the expertise of highly skilled technicians and can be time-consuming or resource-intensive. If the selection/screening strategy is straightforward, the steps of mutagenesis/recombination in vitro may account for nearly all the time and effort spent on a directed evolution project.

Notably, several in vivo mutagenesis approaches have been described, examples including the use of mutator strains and enhancing mutation rates in wild-type cells by chemicals or radiation (Long-McGie et al., 2000; Selifonova et al., 2001; Trower, 1996). These techniques rely on culturing cells, normally bacteria, and therefore do not involve substantial expenses or extensive personnel training.

However, a broader utility of mutator strains and condition-induced mutagenesis is hampered by the indiscriminate nature of mutations, which affect both target sequences and the host cell genome with the probability directly proportional to the nucleic acid length. Because cellular genomes comprise a number of indispensable genes and are several orders of magnitude larger than usual directed evolution targets, the maximal allowed mutation rate is limited by the host tolerance. As a consequence, only moderate mutation rates are available to an artisan willing to modify a protein or a nucleic acid, which necessitates the use of large pools of cells and/or extended mutagenesis times. Furthermore, if the search for improved variants is based on the cell survival, growth rate or morphology, advantageous mutations in the target sequence may be masked by disadvantageous changes in the genetic background of the host, thus reducing the efficiency and accuracy of the selection/screening procedure.

Concerning the second challenge for the art of directed evolution, many methods, such as phage displays, ribosomal displays, cell-surface displays, mRNA display, SELEX, and others, utilize conceptually simple binding procedure to select for proteins or nucleic acids with improved affinities to given ligand. In contrast, only few techniques have been reported for changing enzyme properties. There are reports where phage display and SELEX technologies have been adapted for evolving some enzymatic activities; however, the range of catalytic reactions which can be selected for is limited (see e.g. (Forrer et al., 1999; Wilson and Szostak, 1999). Similarly, the in vitro compartmentalization method, developed by Griffith et al. for evolving nucleic acid modification enzymes (Tawfik and Griffiths, 1998), requires elaborate in vitro manipulations when applied to other types of enzymes (Griffiths and Tawfik, 2003).

Expressing target genes in bacteria and screening/selecting for desired enzymatic activities is one of the most versatile approaches for evolving enzymes with improved properties (e.g. (Cohen et al., 2001). The use of mutagenesis in vivo is extremely advantageous for this group of methods, because (in addition to the aforementioned problems of in vitro diversification techniques) the efficient delivery of large nucleic acids libraries into living cells constitutes a major methodological challenge.

Since existing methods of mutagenesis in vivo also suffer of serious limitations, there is a great need for a rapid, non-laborious, inexpensive method for generating diverse populations of target molecules in vivo, which could be used for changing enzyme properties in a required fashion. Toward this end, the present invention discloses the use of the erroneous nature of RNA-dependent nucleic acid synthesis for the purpose of directed evolution.

As discussed above, in vitro methods may suffer of several limitations, such as being expensive and resource-intensive and requiring skills of highly-trained personnel.

### SUMMARY

In an aspect, this invention utilizes the high mutation rate and adaptability of an RNA-based biological entity (e.g. virus) as a driving force for directed evolution of target sequences. Indeed, replication of RNA genomes is catalyzed by polymerases lacking proofreading function, which makes RNA copying an intrinsically erroneous process (Domingo et al., 2001). Importantly, the novel method for directed evolution has a substantially higher theoretical limit for the maximal allowed mutation rate, than in the existing methods for mutagenesis in living cells, because RNA genomes are much smaller than cellular DNA genomes. This enables an accelerated discovery of improved variants using moderate numbers of the host cells.

One object of this invention is a method for changing a target nucleic acid sequence. The method is mainly characterized by what is stated in the characterizing part of claim 1.

One further object of this invention is a living cell system. The living cell system is mainly characterized by what is stated in the characterizing part of claim 27.

One still further object of this invention is a kit for changing a target nucleic acid or protein sequence. The kit is mainly characterized by what is stated in the characterizing part of claim 31.

Many RNA-based systems can be suitable for practicing the new method of directed evolution. For the purpose of this invention, it may be advantageous to use an RNA virus. Both true riboviruses, whose life cycle proceeds entirely on the RNA level, and so-called reverse-transcribing viruses, which alternate between RNA and DNA genomic forms throughout their life cycles, are acceptable formats. However, in other embodiments, one can make use of essentially any RNA-based organism or system, including RNA virus-like particles, RNA plasmids, viroids, or other RNA-based autonomous genetic elements. According to a preferred embodiment of the invention the RNA based system is an RNA bacteriophage which belongs to Cystoviridae family, preferably the bacteriophage is selected from the group of φ6, φ7, φ8, φ9, φ10, φ11, φ12, φ13 and φ14, most preferably from bacteriophage φ6. The replicable form of the nucleic acid target is contacted with the polymerase in a prokaryotic cell, preferably in a gram-negative bacterial cell, more preferably in a bacterial cell selected from the group comprising Pseudomonas sp., Escherichia sp. and Salmonella sp., most preferably in a cell of Pseudomonas syringae.

A currently preferred embodiment rely on a genetically altered bacteriophage φ6, a dsRNA virus from the Cystoviridae family that infects the bacterium Pseudomonas, in particular P. syringae (Mindich, 1988; Mindich, 1999a).

The target nucleic acid sequence may be homologous or heterologous, in particular it may be heterologous, to the RNA virus or replicon.

The new methods described here are intended primarily for directed evolution of proteins and nucleic acids. Specific applications of the method include but are not limited to improving enzymes, as well as molecules having specific binding and regulatory activities. In other embodiments, the method is used for optimizing RNA stability or codon usage. As with the aforementioned methods of directed evolution, a number of biological entities having RNA genomes will be appropriate systems for the use within this methodology. For example, at least some ssRNA viruses are known to replicate their genomes via dsRNA intermediates (Buck, 1996). However, for the ease of obtaining dsRNA of sufficient purity and in sufficient amounts it is advantageous to use viruses or other types of replicons with dsRNA genomes.

In yet further aspect, the invention provides a novel method for constructing recombinant dsRNA bacteriophages. The method takes advantage of suicide vectors wherein nucleic acid fragments of interest are operably linked with the sequences sufficient for detectable replication by the viral replication apparatus. The new method is faster and easier than previously described methods for constructing recombinant dsRNA bacteriophages, which involve in vitro packaging of procapsids particles (Poranen et al., 2001) or propagating genetically modified bacteriophages in host cells stably transformed with the plasmid expressing target genes (Mindich, 1 999b) and references therein).

In the currently preferred embodiment said suicide vector is a DNA plasmid that is delivered into a cell containing functional viral replication apparatus. The plasmid can not be stably propagated within said cell (definition of a suicide vector), but can be transiently transcribed by a DNA-dependent RNA polymerase to yield RNAs replicable by the viral polymerase.

Said replicable RNAs derived from the suicide plasmid contain target nucleic acid sequence, which makes the suicide vector strategy useful for specific embodiments related to directed evolution.

Further features, aspects and advantages of the present invention will be better understood from the description of specific embodiments and examples. It should be understood, however, that the description and the examples are given by the way of illustration only, not by the way of limitation. Various changes and modifications within the spirit and the scope of the invention will become apparent to those skilled in the art from the following text. Furthermore, citation of a reference throughout the entire patent text shall not be interpreted as an admission that such is prior art to the present invention.

### BRIEF DESCRIPTION OF THE FIGURES

The foregoing text, as well as the following description and appended claims, will be better understood when read in conjunction with the appended figures, in which:

FIG. 1 shows schematically how recombinant RNA replicons are generated using suicide plasmid strategy. The example depicts constructing carrier-state Pseudomonas syringae cells that contain recombinant φ6 virus expressing beta-lactamase gene (φ6-bla).

FIG. 2 depicts:

(A) Agarose gel electrophoresis of total RNA from the following strains: K, Km-resistant HB10Y(φ6-npt); A0, Amp-resistant HB10Y(φ6-bla); HB, non-infected HB10Y. Lane φ6, dsRNA segments L, M and S extracted from the wild-type φ6 (positions indicated on the left along with the positions of P. syringae 23S and 16S rRNAs). Mk, dsDNA markers. Marker lengths in kbp are shown on the right. White arrowhead shows the new segment, M-bla, which appears in Amp-resistant cells.

(B) RT-PCR analysis with npt- and bla-specific primers was performed using RNA from: K, HB10Y(φ6-npt) and A0, HB10Y(φ6-bla). The reverse transcription (RT) step was omitted in reactions 2 and 5. Different PCR primers were used as specified under the panel. Positions of the npt and bla-specific PCR fragments are marked on the right. dsDNA marker (Mk) lengths are shown on the left.

FIG. 3 shows that φ6-bla carrier cells rapidly adapt to cefotaxime.

(A) 0.2 to 1×10⁷ HB10Y(φ6-bla) carrier state cells were plated onto LB agar containing either 150 μg/ml ampicillin (Amp150) or 50 μg/ml cefotaxime (Ctx50). Ctx resistant colonies appeared after 3 days of incubation at 28° C. No colonies were detected at this time on the sector inoculated with 1×10⁷ HB10Y(pLM254) cells, which contain a plasmid encoding the bla gene.

(B) Schematic diagram of the Ctx adaptation experiment. Cells were cultivated on LB agar containing increasing Ctx concentrations (μg/ml), as shown below petri dishes. 20-40 of the largest colonies were pooled after each passage and used for subsequent rounds of selection.

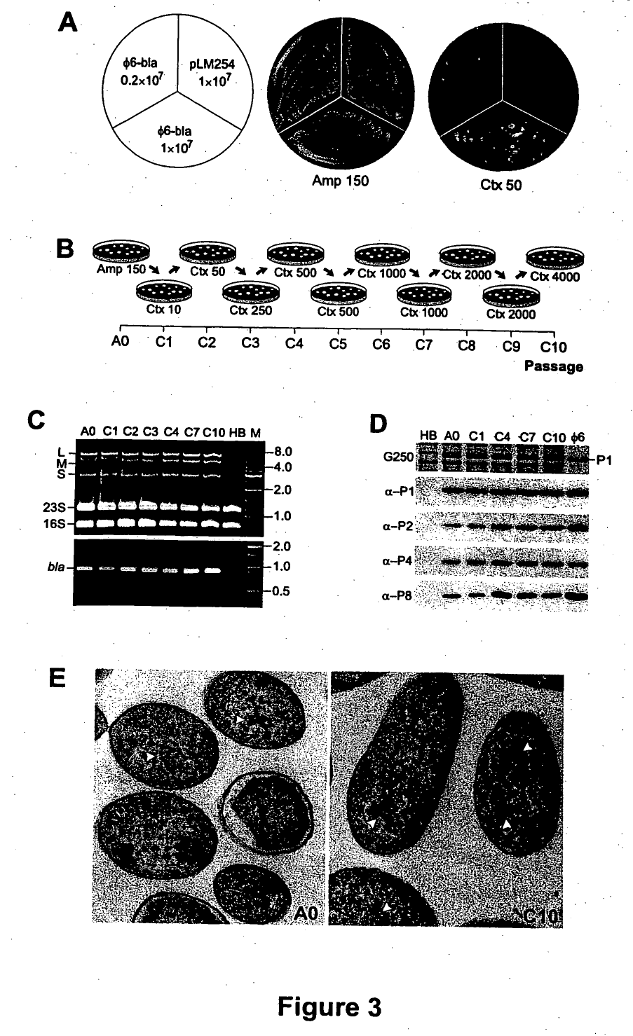

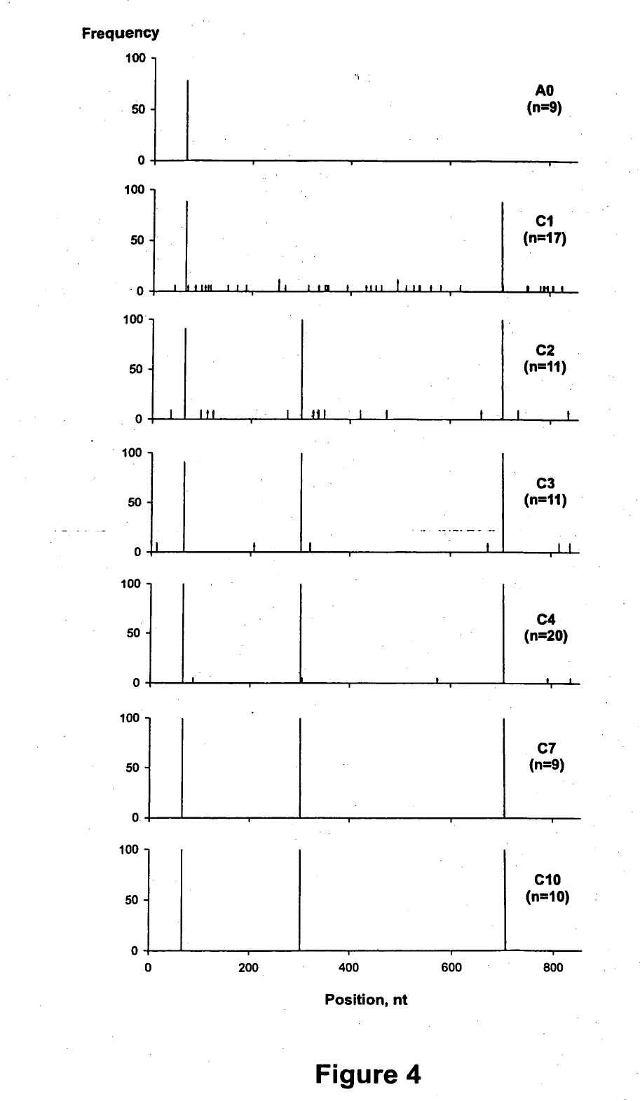

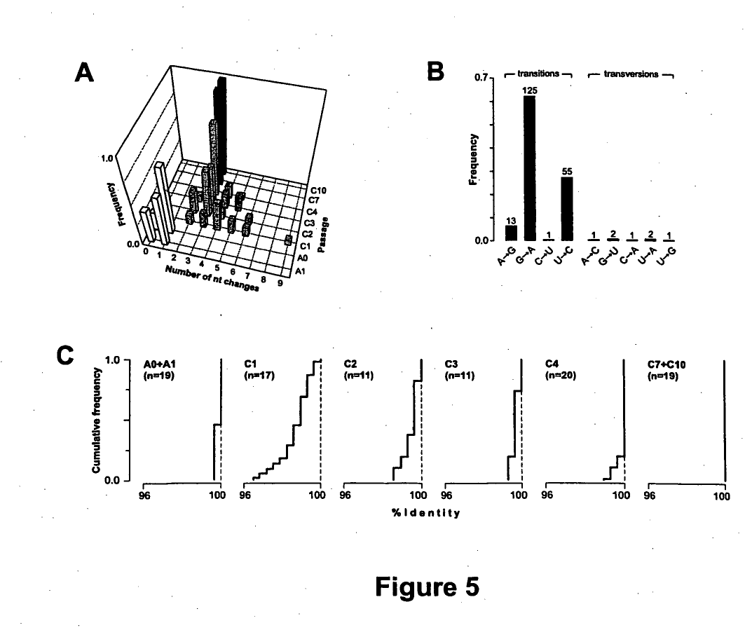

(C) Upper panel, agarose gel analysis of RNA extracted from carrier state cells at passages A0, C1, C2, C3, C4, C7 and C10. HB, RNA from uninfected HB10Y cells. Lower panel, RT-PCR products generated using bla-specific primers. Other designations are as defined in the description of FIG. 2.

(D) SDS-PAGE analysis (Olkkonen and Bamford, 1989) of carrier state cells from different passages (A0, C1, C4, C7 and C10) or purified φ6 virus (φ6). HB, uninfected HB10Y cells. Panel G250, a Coomassie G250 stained gel fragment showing the band of protein P1. α-P1, α-P2, α-P4, and α-P8, immunoblots produced using antibodies specific to corresponding φ6 nucleocapsid (NC) proteins and ECL detection as recommended by Pierce Biotechnology.

(E) Transmission electron micrograph of osmium tetroxide and uranyl acetate stained cell thin sections from A0 and C10 passages taken as described (Bamford and Mindich, 1980). Black arrowhead, enveloped virions; white arrowhead, NC and PC particles.

FIG. 4 depicts changes in the bla sequence population in response to cefotaxime selection.

Graphs show normalized point mutation frequency at indicated nucleotide positions summed for n bla sequences from each passage. Bars corresponding to synonymous nucleotide changes are marked with the circles. Unmarked bars, missense mutations.

FIG. 5 depicts further aspects of population dynamics of bla sequences during adaptation to cefotaxime.

(A) Normalized frequency of bla alleles containing a given number of mutations as a function of passage. White, passages A0 and A1; gray, passages C1 to C4; black, passages C7 and C10.

(B) Distribution of different mutation types in bla sequences from C1, C2, and C3.

(C) Percent identity plots showing genetic variance in bla populations from different passages. Plots (solid lines) are cumulative distribution functions of identities between every pair within n sequences, where the vertical axis represents the fraction of data points with the value as small or smaller than a given identity value. More heterogeneous sequence populations give plots more deviated from the 100% identity asymptote (dashed line). Data for related passages A0 and A1 and also for C7 and C10 were combined to improve statistics. Plots were created in GeneDoc (http://www.psc.edu/biomed/genedoc/).

### DETAILED DESCRIPTION OF THE INVENTION

1. Definitions

Unless explicitly stated otherwise, specific terms used throughout this invention have the following meanings:

The term “bacteriophage” refers to a virus infecting eubacteria or another prokaryotic organism, such as e.g. archaea.

The term “biological activity”, as used herein, refers broadly to various functions and properties of a protein or nucleic acid. Examples of biological activities include but are not limited to catalytic, binding, and regulatory functions.

As used herein, the term “biological entity”, refers to all systems containing nucleic acids capable of multiplication through a template-directed mechanism.

As used herein, the term “carrier-state cells” refers to a cell line or plurality of cells infected by a virus, which can support multiple rounds of the virus genome replication, remaining in a living state for a period of time substantially longer than a typical duration of the virus life cycle.

As used herein, the term “directed evolution”, or sometimes “directed molecular evolution”, refers to a process of intentionally changing properties of proteins or nucleic acids using the algorithm, which comprises one or several rounds of subsequent diversification and selection steps. This algorithm is ascribed to natural evolution by Darwin's theory.

The term “DNA-dependent polymerase” refers to nucleic acid polymerase capable of copying DNA templates. Two types of DNA-dependent polymerases are known, producing DNA or RNA copies of DNA templates. These are referred to as DNA-dependent DNA polymerases and DNA-dependent RNA polymerases, respectively. Also see “polymerase”.

The term “erroneous nature” is used here in reference to template-dependent nucleic acid polymerases lacking proofreading function or when describing the process catalyzed by such polymerases.

The term “nucleic acid sequence”, or sometimes “nucleotide sequence”, refers to an order of nucleotides in an oligonucleotide or polynucleotide chain.

The term “polymerase”, or sometimes “nucleic acid polymerase”, refers to a protein or a protein complex that can catalyze the polymerization of ribo- or deoxyribo-nucleoside triphosphates into a polynucleotide chain.

The term “protein sequence”, or sometimes “amino acid sequence”, refers to an order of amino acid residues in a peptide or protein chain.

The term “proofreading”, as used herein, refers to the capacity of certain polymerases to remove nucleotides incorrectly incorporated into a growing nucleic acid chain thus increasing fidelity of the template copying process. In template-directed synthesis, nucleotide incorporation into nucleic acid chain is considered incorrect if against the base complementarity rules by Watson and Crick. Polymerases of the present invention are characterized by the lack or deficiency of the proofreading activity, which enhances the mutation rate and generates sequence diversity in the target population.

As used herein, the term “ribovirus” refers to an RNA virus whose life cycle proceeds entirely on the level of RNA and does not normally include a DNA phase. Riboviruses include viruses with positive- and negative-sense single-stranded (ss) RNA genomes as well as double-stranded (ds) RNA viruses. A preferred embodiment of this invention deals with dsRNA viruses from the Cystoviridae family, also referred to as “cystoviruses”. Also see “RNA virus”. The dsRNA virus is preferably a bacteriophage selected from the group comprising φ6, φ7, φ8, φ9, φ10, φ11, φ12, φ13 and φ14, most preferably it is bacteriophage φ6.

As used herein, the term “reverse-transcribing virus” refers broadly to a virus whose life cycle necessarily includes both RNA and DNA phases. The name of the group derives from the process of “reverse transcription” used by these viruses wherein RNA molecules are used as templates to produce DNA copies. Two types of reverse-transcribing viruses are known, “retroviruses” and “pararetroviruses”. Retroviruses encapsidate their genomes in the form of RNA but use DNA intermediates when multiplying in infected cells. Pararetroviruses encapsidate DNA genomes but use RNA intermediates when multiplying in infected cells.

The term “ribozyme” refers to an RNA molecule with detectable catalytic activity. Various natural and artificial ribozymes possessing diverse catalytic activities have been described in the previous art (Bittker et al., 2002b; Doudna and Cech, 2002; Jaschke, 2001).

The term “RNA virus” refers to viruses having RNA genomes.

As used herein, the term “RNA-based autonomous genetic element” refers generically to biological entities containing RNA genome but distinct from RNA virus. RNA-based autonomous genetic elements include but are not limited to RNA virus-like particles, viroids, and RNA plasmids. Another term sometimes used in the literature to refer to RNA-based autonomous genetic elements is “RNA subviral agent”. Also see definition of “biological entity”.

The term “RNA-based organism”, as used herein, refers generically to RNA viruses and RNA-based autonomous genetic elements defined above. Because all RNA organisms are capable of replicating their genomes under appropriate conditions, the term “RNA replicon” is used herein in reference to RNA organisms and derivatives thereof to emphasize this capability.

The term “RNA-dependent polymerase” refers to a nucleic acid polymerase capable of copying RNA templates. Two types of RNA-dependent polymerases are known, producing RNA or DNA copies of RNA templates. These are referred to as “RNA-dependent RNA polymerases” (“RdRP”) and “RNA-dependent DNA polymerases” (“RdDP”, better known as reverse transcriptases), respectively. Also see “polymerase”.

As used herein, the term “screening” refers to procedures wherein variants having preferred properties are identified and/or picked from a target population manually or using an automated process.

The term “selection” is used herein in two contexts. In a specific context, “selection” refers to procedure wherein different variants of a target population compete with each other so that only the fittest variants are retrieved, whereas less fit members of population are lost.

This can be also defined as “bona fide selection”. In a more general context, “selection” refers generically to all procedures (including “screening” and “bona fide selection”) wherein a fraction of variants is withdrawn from a target population for further use.

As used herein, the terms “target” or “target molecule” refer to a protein or nucleic acid that is subjected to the methods of this invention, which are designed for changing nucleic acid and proteins. Plurality of target molecules comprising one or many distinct variants is sometimes referred to as “target population”. The length of a target nucleic acid can be from about 20 bases, preferably from about 50 bases to 15 kilobases, more preferably it is from 50 bases to 5 kilobases, still more preferably from 300 bases to3 kilobases.

“Heterologous target sequence” refers here to a target sequence from any possible origin except from the RNA-based biological entity (e.g. RNA virus), which is used in the replication of the target sequence. Homologous target sequence” refers here to a target sequence from the RNA-based biological entity (e.g. RNA virus), which is used in the replication of the target sequence.

“Detectable replication” refers here to the replication of the nucleic acid target detectable by any standardly available molecular biology method.

“A living cell” refers here to a cell supporting the replication of an RNA-based biological entity, such as RNA virus or other RNA replicon. The living cells may belong to prokaryotes. They may be bacteria, preferably gram-negative bacteria, more preferably bacteria selected from the group comprising Pseudomonas sp., Escherichia sp. and Salmonella sp., most preferably Pseudomonas syringae. The living cell may also be a eukaryotic cell, such as mammalian, insect, plant or yeast cell.

As used herein, the term “suicide vector” or a more specific term “suicide plasmid” refer to, respectively, vector/plasmid that can not be stably maintained within given cell line but can direct transient gene expression.

Other terms are explained in the text or used according to the common practices of the art.

2. Directed Evolution

2.1. General Considerations

In the first aspect, this invention provides a method for changing nucleic acids and proteins.

Replication of RNA genomes is catalyzed by RNA-dependent polymerases that lack proofreading function. This makes RNA copying an intrinsically erroneous process. As a specific example, relevant to preferred embodiments of this invention, the per-nucleotide mutation rate for dsRNA bacteriophage φ6 has been estimated at ˜1×10⁻⁵ to 2.7×10⁻⁶ depending on the method used (Chao et al., 2002; Drake and Holland, 1999).

In addition to the high mutation rate, many RNA genomes are capable of homologous and/or non-homologous recombination, which further contributes to the genetic diversity (Domingo et al., 2001; Miller and Koev, 1998; Negroni and Buc, 2001). Notably, genomes of dsRNA bacteriophages from the Cystoviridae family have been reported to recombine with a detectable efficiency (Onodera et al., 1993; Onodera et al., 2001; Qiao et al., 1997; Qiao et al., 2000).

The quasispecies theory describes populations of RNA replicons as clouds, or swarms, of distinct but closely related genotypes (Domingo et al., 1996; Eigen, 1996). Such organization allows the rapid adaptation to new environments, since a number of potentially advantageous mutations are already present in the population at the onset of selective pressure.

Therefore, high mutation and recombination rates are likely reason of the remarkable evolutionary success of RNA viruses. Many RNA viruses, including HIV and hepatitis C virus, are known to efficiently escape host immune responses and medical treatment by promptly accumulating resistant mutants (Domingo et al., 1997; Farci et al., 2000; Harrigan and Alexander, 1999). With continually emerging new strains and even species (Fouchier et al., 2003; Marra et al., 2003; Nichol et al., 2000), RNA viruses cause over 75% of all viral diseases and constitute an overwhelming majority of all viral species (Domingo et al., 2001).

It is within the scope of this invention to utilize the high evolutionary potential of RNA replicons for changing properties of target nucleic acids and proteins. The relevant method comprises the steps of:
a) providing input nucleic acid target in a form replicable by a polymerase devoid of the proof-reading function;
b) contacting said replicable form of the nucleic acid target with said polymerase under conditions sufficient for template-directed nucleic acid synthesis in a living cell;
c) recovering nucleic acid synthesis products, whose nucleotide sequence differs from said input target sequence by at least one nucleotide.

It is obvious that the above method can be used in its general form for introducing advantageous, neutral and/or disadvantageous changes into the nucleic acid sequence of interest (nucleic acid target).

However, in the currently preferred variation of the method, said recovering of modified nucleic acid synthesis products is performed after an appropriate selection/screening procedure, so that only advantageous changes are recovered. In this preferred form the method is intended for directed molecular evolution. This method variation employs an optimization algorithm comprising the steps of:
a) providing input nucleic acid target in a form replicable by a polymerase devoid of the proof-reading function;
b) contacting said replicable form of the nucleic acid target with said polymerase under conditions sufficient for template-directed nucleic acid synthesis in a living cell;
c) selecting or screening nucleic acid synthesis products based on their properties;
d) recovering nucleic acid synthesis products, whose properties are deemed superior to said input nucleic acid target.

In some embodiments, it will be sufficient to carry out only one round of the above optimization algorithm to improve target sequence to a sufficient extent. However, the method users will often find it more advantageous to perform two or more rounds of optimization. Indeed, the evolution of the TEM beta-lactamase sequence described in the Examples was carried out using at least two optimization rounds (passages).

An important aspect of the method described above is the nature of the target. The strategy used by the method dictates the physical nature of the target to be a nucleic acid, preferably RNA, a usual template for polymerases lacking proofreading function. However, many nucleotide sequences can be translated into amino acid sequences, which makes the present invention broadly related to changing/improving both nucleic acids and proteins.

2.2. Preferred Formats

Specific embodiments of the above-described method for changing nucleic acid and proteins as well as the currently preferred method for directed evolution, may differ with the respect to the formats used.

Viral RNA Vectors

It is essential for the changing/evolving procedure that the nucleic acid target is provided in a form replicable by a polymerase devoid of the proofreading function. In most embodiments, this step is realized through linking the target with determinants required for detectable replication by said polymerases.

In the selected formats, target is integrated within RNA replicons, thus allowing replication of the target by an appropriate RNA-dependent polymerase. It may be advantageous for many applications to choose RNA viruses as RNA replicons. In this case, integrated target is replicated as a part of viral genome by the virus-encoded polymerase, preferably RNA-dependent polymerase. Previous experiments where RNA viruses were used as vectors for heterologous sequence inserts demonstrates feasibility of this approach. For example, alphaviruses, retroviruses and some (−)RNA viruses are used as vectors for gene therapy and gene expression application (Palese, 1998; Robbins et al., 1998). Similarly, several RNA viruses infecting plants may also be used as vectors (Lindbo et al., 2001).

Although some embodiments of the method can rely on single-stranded RNA viruses, it may be advantageous for many applications to select viruses that have double-stranded RNA genome. dsRNA resist nuclease degradation better than ssRNA, which makes it easier to purify sufficient amount of intact dsRNA than that of ssRNA. Examples of dsRNA viruses include members of the Cystoviridae, Reoviridae, Totiviridae, Partitiviridae, Birnaviridae and Hypoviridae families. Because of the economical and convenience reasons it may be advantageous to use viruses from the Cysto-, Toti- and Partitiviridae families, which infect prokaryotes and lower eukaryotic organisms such as bacteria, yeast and other fungi. Bacteriophage φ6 and its relatives (φ7 through φ14) infecting gram-negative bacteria and Saccharomyces cerevisiae viruses L-A and L-BC, that have been also known under the name of “virus-like particles”, are amongst the most obvious choices.

In the currently preferred embodiment, target gene is integrated within the genomic RNA of a dsRNA bacteriophage from the Cystoviridae family (a cystovirus). An important advantage of an RNA bacteriophage over animal or plant RNA viruses is the low cost and relative ease of propagation. Furthermore, bacteriophages generally have shorter life cycles, which helps to reduce the time of the experiment.

As a specific example of the dsRNA bacteriophage format, target gene can be integrated into the M segment of the cystovirus φ6 and replicated by the φ6-encoded RNA-dependent RNA polymerase. In further embodiments, other members of the Cystoviriae family, from φ7 through φ14 (Mindich et al., 1999), can be used as vectors for target sequences and also as polymerase source. Furthermore, any of the three genomic segments L, M and S, typical for the Cystoviridae, can be used for integrating the target sequence.

Furthermore, it is known that at least some cystoviruses can tolerate substantial genome rearrangements, which can be manifested in the form of shortened or extended genomic segments, or a change in the segment number. For example, variants of φ6 containing 1, 2 or 4 genomic segments have been described (Onodera et al., 1995; Onodera et al., 1998). These modified cystoviruses are also within the scope of this invention, as they can be more advantageous RNA vectors than the wild-type cystoviruses.

It has been shown that the synthesis of cystoviral RNA is catalyzed by so-called polymerase complex that includes proteins P1, P2 (catalytic subunit), P4, and P7 (Mindich, 1999a; Mindich, 1999b). The polymerase complex also serves as a container for genomic RNA. All polymerase complex proteins are encoded on the segment L. Earlier studies have also demonstrated that bacterial cells expressing cDNA of the L segment accumulate functional polymerase complex particles (Mindich, 1999b). Therefore, some embodiments may involve the use of cystovirus derivatives whose L segment encodes for the polymerase complex, whereas additional segment(s) are used for incorporating nucleic acid targets. In alternative embodiments, proteins of the polymerase complex can be produced from cDNA, which can be introduced into bacterial cell for example in the form of a DNA plasmid. In this case, the entire genetic capacity of the polymerase complex (˜15 kb) can be used by RNA segment(s) encoding the evolution target(s).

It is currently preferred feature that the RNA virus vector used is propagated in the form of carrier state cells. This type of viral infection does not destroy most of the infected cells, thus effectively extending time of the target gene expression. Clearly, all formats where virus is not lethal for the infected cell will be particularly useful for the protein evolution projects.

In the currently preferred embodiment, recombinant bacteriophage φ6 is propagated within carrier-state bacteria Pseudomonas syringae. Because at least some of the related cystoviruses have been shown to infect Escherichia coli and Salmonella typhimurium (Hoogstraten et al., 2000; Mindich et al., 1999; Qiao et al., 2000), additional embodiments of this invention will be based on the use of carrier-state gram-negative bacteria containing a recombinant cystovirus selected from the group of φ6, φ7, φ8, φ9, φ10, φ11, φ12, φ13, and φ14.

In further specific embodiments, non-lethal infection can be achieved by using special cell lines, weakened (attenuated) virus strains, or both. As an example of the first strategy, mutants of P. syringae cells are known that form carrier state cells after being infected with the wild-type φ6 virus. Attenuated viruses can be selected as naturally occurring mutants or engineered artificially. In some cases it will be sufficient to substitute a part of viral genes with the target sequence to obtain an attenuated virus. Interestingly, non-lethal infection is typical for the normal life cycles of several viruses. The examples include above-mentioned yeast totiviruses L-A and L-BC.

Non-viral RNA Vectors

Although the use of virus-based vectors is advantageous for many applications, some embodiments of the directed evolution method may use non-viral vectors. One example of this strategy is to use specific elements that are replicated in nature by viral RNA-dependent RNA polymerases, such as diverse defective interfering (DI) elements and satellite RNAs. Specific examples include small RNAs multiplied by the RdRP of the coliphage Qβ and toxin-encoding satellites of the yeast L-A virus (M1, M2, and others) (Brown and Gold, 1995; Wickner, 1996).

Another example of non-viral vectors would be the use of autonomous genetic elements found for example in fungi and plants. S. cerevisiae strains often contain single-stranded replicons called 20S RNA and 23S RNA. Of these, 20S RNA is an apparently naked RNA replicon (with a dsRNA form called W) encoding an RNA polymerase. 23S RNA also encodes an RNA polymerase and has a dsRNA form called T (Wickner, 1996). Furthermore, some plants, such as rice, are infected by extensive dsRNA elements, referred to as “RNA plasmids” or “endomaviruses” by different authors (Gibbs et al., 2000). These elements encode their own RdRP and seem to lack coat proteins. Many RNA replicons of the non-virus origin normally do not destroy the infected cell, which can be an advantageous feature as discussed above.

Polymerase Sources

In the aforementioned embodiments, target nucleic acid, integrated into viral or non-viral RNA vector, is replicated by an RNA-dependent polymerase. It will be obvious for those skilled in the art that said polymerase can be provided in any number of ways. In some embodiments, the polymerase will be encoded by the RNA replicon containing the nucleic acid, whereas in other embodiments the polymerase will be encoded by another RNA replicon co-infecting the host cell.

In yet further embodiments, the polymerase can be encoded by DNA, which can be of chromosomal, plasmid, viral, transposon or other origin. An example of this format was discussed above for cystovirus-based vectors. In another specific embodiment, target sequence can be incorporated into viroid RNA and the replication of the genetically altered viroid RNA is probably carried out by cellular RNA polymerase II, operating in this case in the RNA-dependent mode (Lai, 1995). In other embodiments, viral polymerase genes can be introduced in a DNA form into the host cell and expressed using cellular transcription and translation apparatus.

Delivery Methods

Another important aspect of the methods for changing/evolving biological molecules is the procedure used for bringing nucleic acid targets in contact with the polymerase lacking proofreading function.

In a specific embodiment of this invention, this task can be accomplished by contacting a replicable form of the nucleic acid target with said polymerase within living cell. For this purpose, both target and the polymerase have to be delivered into the host cell.

Different delivery methods can be used in different embodiments, ranging from delivery through virus infection, transformation (in bacteria), transfection (in eukaryotic cell lines), electroporation, lipofection, ballistic methods, agroinfiltration, microinjection etc. Description of these and other delivery methods can be found elsewhere.

In the currently preferred embodiment, illustrated in the Example 1, bacteriophage φ6 RdRP is delivered into the host P. syringae cell using virus infection. The heterologous sequence is delivered either through virus infection (as in the φ6-npt case) or in the form of a suicide DNA plasmid using electroporation (as in the φ6-bla case).

In many embodiments, it may be advantageous to deliver RNA replicons containing marker genes. Such marker genes can be very useful to distinguish between cells that contain RNA replicon from the rest of the cells. Indeed, currently available delivery methods may not be 100% efficient, in that only a fraction of the treated cells usually receive the RNA replicon encoding the nucleic acid target. Examples of marker genes may include antibiotic or toxin resistance genes, genes encoding enzymes of amino acid or nucleotide metabolism, or genes encoding fluorescent proteins. Although in some embodiments the marker gene can be equivalent to the evolution target, other embodiments may use marker genes that are distinct from the evolution targets. In the latter case, it is advantageous to ensure a physical linkage between said marker and target. In a preferred embodiment, said linkage is achieved through encoding both marker and target on a single RNA segment.

2.3. Preferred Applications

The directed evolution methods of this invention can be preferably used to modify various properties of nucleic acids and proteins, as explained below.

Evolving Enzymes

In a specific embodiment, gene encoding an antibiotic-degrading enzyme (ampicillin-specific β-lactamase) is inserted into RNA virus genome. After an appropriate selection procedure a gene having modified sequence is recovered, that encodes the enzyme having altered antibiotic specificity (hydrolyzes cefotaxime in addition to ampicillin). The modified antibiotic resistance genes can be useful as markers or reporters. Alternatively, this RNA-replicon based evolution procedure can be used to assess the probability of developing an antibiotic resistance to new antibiotics in pathogenic bacterial strains, as explained earlier (Orencia et al., 2001).

This invention provides in particular a suicide vector, which comprises a beta-lactamase gene operably linked with determinants essential for detectable replication by the RNA-synthesis apparatus of a Cystoviridae member, preferably bacteriophage φ6.

This invention provides also a genetically modified cystovirus, which comprises a beta-lactamase gene conferring resistance to one or several antibiotics of the penicillin group, preferably ampicillin. In particular, this invention provides a genetically modified cystovirus, which comprises a beta-lactamase gene conferring resistance to one or several antibiotics of the cephalosporin group, preferably cefotaxime.

Furthermore, this invention provides carrier-state cells, which comprise the mentioned cystoviruses.

In additional embodiments, the directed evolution method can be generally used to create new catalysts, including diverse protein enzymes and ribozymes, or improve already existing ones. Several parameters can be subjected to directed evolution process, including the use of modified substrates, substrate affinity and turnover, pH, ion strength, or temperature optima, enzyme behavior with respect to inhibitors and activators, and so on.

In a specific embodiment where RNA catalysts (ribozymes) are targets for directed evolution, these are physically incorporated into RNA replicon, thus providing a link between genotype and phenotype. On the other hand, in other embodiments, designed for evolving protein catalysts, RNA replicons encode target proteins. In this latter case, the link between genotype and phenotype is provided by virtue of co-occurrence of RNA-replicons and the cognate protein products within the same cell. Thus, by selecting a cell expressing improved enzymatic activity one will also select the gene encoding the improved enzyme.

An obvious requirement imposed on the directed evolution method of this invention is the need for selection or screening procedure, which is essential to recover improved variants after the sequence diversification step. A number of examples where such selection/screening procedure was possible have been discussed elsewhere.

It may be advisable to devise a selection procedure if the enzyme can substantially contribute to the cell metabolism. Examples of this type include enzymes of amino acid, nucleotide and co-enzyme metabolic pathways, as well as hydrolases of different biopolymers. In some embodiments, it may be advantageous to perform selection for such activities using auxotrophic or otherwise deficient host cells. Furthermore, enzymes essential for cell survival under specific conditions such as those inactivating toxins, heavy metals, cell growth inhibitors should be evolved via appropriate selection procedure rather than screening.

On the other hand, enzymes that can be detected by a color or fluorescent assay will be perhaps easier to evolve using manual or automated screening, e.g. by using different detection units together with image recognition algorithms or alternatively by cell sorting methods such as fluorescence assisted cell sorting (FACS).

While the currently preferred embodiments of this invention deal with single enzymes, other embodiments may be focused on a simultaneous evolution of a group of enzymes catalyzing several reactions, e.g. interdependent reactions constituting a methabolic pathway or a part thereof. (Indeed, directed evolution methods have been successfully applied to metabolic engineering; see (Zhao et al., 2002) and references therein). In this case different genes can be encoded by a single RNA replicon or alternatively provided as several co-existing RNA replicons. In the specific embodiment where multiple enzymes are evolved using φ6 system, it may be advantageous to use the entire coding capacity of at least M, preferably both M and S, most preferably all three genome segments, L, M and S.

Evolving Regulatory Molecules

A specific embodiment of the above methods can be used for evolving regulatory molecules. As in the case concerning enzyme evolution, the method can be directed to either engineering novel regulatory activities or improving existing ones. In some cases, regulatory molecules can be proteins or RNAs that activate or inhibit enzymatic activities through direct interaction with the enzyme. Examples of this class of molecules include e.g. different RNase and polymerase inhibitors (Jeruzalmi and Steitz, 1998; Pasloske, 2001).

In other cases, regulatory protein or RNAs can modulate gene expression exerting activation or inhibition effects on the transcription, translation, or other levels of gene expression. This class of regulators includes different activators and repressors that interact with regulatory regions, such as gene promoters and terminators, as well as mRNA untranslated regions. Examples of regulatory proteins include catabolite activator protein (CAP), Lac repressor (Lacd), bacteriophage lambda repressors CI and Cro, eukaryotic transcription factors such as GAL4, mRNA cap- and iron-responsive element binding proteins, and many others. In addition many regulators interact with basal factors involved in transcription or translation as discussed previously (Lemon and Tjian, 2000; Sachs and Buratowski, 1997). At the RNA level, examples of regulatory elements include translation enhancers, such as internal ribosomal binding sites (IRES) and diverse stem-loop/tRNA-like/pseudoknot structures found in RNA viruses (Gallie and Walbot, 1990; Leathers et al., 1993; Olsthoorn et al., 1999; Sachs, 2000; Vagner et al., 2001; Zeenko et al., 2002). Further examples include regulatory elements controlling mRNA stability and efficiency of translation both in cis (e.g. iron-responsive elements (IRE) (Theil, 1993)) and in trans (e.g. recently discovered small regulatory RNAs, also known under the names of mRNAs and stRNAs (Grosshans and Slack, 2002)).

Regardless of the regulation level, a preferred protocol for evolving regulatory molecules involves selection or screening for enzymatic (or other) activity that is affected by the regulator. If the evolution target is an activator, cells showing the highest enzymatic (or other) activity are selected. In contrast, cells showing the lowest activity are selected when it is necessary to improve an inhibitor.

Evolving Molecules with Specific Binding Activities

In further embodiments, the evolution method of this invention can be used to develop or modify specific binding activities of proteins or RNAs. As in the case with enzymatic and regulatory activities, evolution of RNA molecules having specific binding properties will require that the binding molecule is a physical part of a larger RNA replicon. And again, proteins with specific binding activities are produced from genes encoded by RNA replicons.

Selection for binding activities may require special experimental formats, involving displaying binding molecules for binding with immobilized or immobilizable ligands. In a specific embodiment, protein having specific binding activity is displayed on the surface of the cell containing RNA replicon, which encodes for the binding protein. Cells expressing desired variant of the protein can be separated from the pool of cells expressing other variants of the protein or expressing no protein at all using an affinity selection procedure.

In an alternative embodiment, proteins having an affinity to a given ligand are displayed on a virus particle. The virus particle occludes the RNA replicon encoding the protein displayed, thus providing a genotype-phenotype link. Notably, the virus may or may not be the source of the polymerase activity required for the (erroneous) propagation of the RNA replicon within host cell. In any case, the virus particles bearing the specific binder on the surface are selected from the pool of irrelevant virus particles using affinity purification based on the interaction with the ligand.

In other embodiments, more specific strategies of selection can be used, depending on the nature of the binding molecule. For example, if the binding molecule is a part of a signal transduction pathway (such as cellular receptors or receptor-binding proteins), screening or selection for a specific cellular response triggered by the pathway can be used for evolving the binding activity.

Evolving Molecules with Other Activities

Yet in further specific embodiments, other biological activities can be improved using the evolution method of this invention. As an example, the procedure can be applied to the green fluorescent protein (GFP) originating from a jellyfish (van Roessel and Brand, 2002). Wild-type GFP is excited by a blue part and emits in the green of the spectrum. A number of GFP mutants with different spectral characteristics have been created using different diversification and screening/selection procedures. Some of the modified GFP variants are used as markers in cell biology and related fields. Using the evolution strategy of this invention, GFP gene can be propagated in a specific embodiment within an appropriate RNA replicon. Some of the appearing GFP mutants can differ from the wt protein in their excitation or/and emission spectra. The cells producing altered GFP (and therefore containing RNA replicons with the mutant GFP gene) can be detected either by eye or using an automated procedure such as e.g. FACS.

The above procedure may be used in other embodiments for evolving other fluorescent and pigment-binding proteins, as well as certain enzymes generating colored or fluorescent products and/or using colored or fluorescent substrates.

Other Utilities

In additional embodiments, the directed evolution method can be employed for specific uses such as improving RNA stability, translation efficiency or codon usage. In this case a target RNA molecule encoding for a detectable biological activity is integrated into RNA replicon and the expression level of the encoded product is scored using an appropriate detection method. Some mutations generated during the propagation of the RNA replicon can increase the expression level of the product without affecting its biological activity target. It is expected that among such mutations can be changes increasing RNA stability against nuclease degradation, translation efficiency and the changes of rare codons to more commonly used ones.

3. A Living Cell System for Changing a Target Nucleic Acid Sequence

One further object of this invention is a living cell system for changing a target nucleic acid sequence. The system comprises:
a target nucleic acid sequence operably linked with determinants essential for replication by an RNA synthesis apparatus of an RNA virus or another RNA replicon;
a living cell capable of supporting the replication of the RNA virus or other RNA replicon; and
a selection/screening procedure for selecting/screening a change in the properties of the nucleic acid synthesis products.

4. Kit for Changing Nucleic or Protein Sequences

One further object of this invention is a kit for changing nucleic acid or protein sequences. The kit comprises one or more, preferably at least two of the following items:
a) a vector for transient expression of target nucleic acid in preselected cells that either are carrier-state or can be transformed into carrier state and/or
b) a genetically modified virus into where the target nucleic acid can be introduced; and/or
c) cells that either are carrier-state or can be transformed into carrier state.

The vector is preferably a suicide vector.

The following Examples provide further illustrations of various aspects and embodiments of the present invention. A skilled artisan will appreciate that specific details can be modified without departing from the scope of the invention.

### EXAMPLES

### Example 1

### Introducing Heterologous Sequences Into the Genome of dsRNA Virus φ6 and Creating Carrier-state Host Bacteria

1.1. Bacterial Strains and Plasmids

Escherichia coli DH5α was used as a host for plasmid propagation and gene engineering. Plasmid pEM35 was produced by inserting the neomycin phosphotransferase (npt) cassette from pUC4K (Pharmacia) at the PstI site of pLM656 (Olkkonen et al., 1990). The correct plasmid encoding the φ6 M segment with the inserted npt gene in the sense orientation was selected using restriction analysis. To construct pEM37, the TfiI-XbaI fragment, containing the φ6 M segment, was excised from pLM656, the ends were filled in using the Klenow fragment of DNA polymerase I, and the blunt fragment was inserted into the pSU18 vector (chloramphenicol resistance marker; (Bartolome et al., 1991)) at HindIII-XbaI sites. To produce pEM38, the β-lactamase (bla) gene was amplified from pUC18 using the primers 5′-TTCACTGCAGATGCATAAGGAAGCATATGAGTATTCAACATTTCCGT-3′ (SEQ ID NO:1) and 5′-CAAACTGCAGAAGCTTACCAATGCTTAATCAGTGAGGCA-3′ (SEQ ID NO:2) and Pfu DNA polymerase (Stratagene). The resulting PCR fragment was inserted at the PstI site of pEM37 in the sense orientation.

1.2. Constructing φ6-npt Carrier-state Cells

The infection of Pseudomonas syringae HB10Y with the wild-type φ6 culminates in cell lysis and release of viral progeny (Mindich, 1988). However, when the kanamycin resistance marker npt was inserted into φ6 M segment, it was possible to select carrier state bacteria on Km-containing medium (Onodera et al., 1992).

We repeated this experiment to obtain a Km-resistant strain HB10Y(φ6-npt). Briefly, purified recombinant φ6 procapsids (PCs) were packaged in vitro with recombinant m⁺ (single-stranded sense copy of φ6 M segment) containing the npt gene (T7 transcript from pEM35 treated with XbaI and mung bean nuclease) and the wild-type l⁺ and s⁺ (single-stranded sense copies of L and S). The packaged ssRNAs were converted into dsRNAs using PC replication in vitro and the particles were coated with φ6 P8 protein to produce infectious nucleocapsids (Bamford et al., 1995). These were used to produce recombinant virus plaques on a P. syringae HB10Y lawn. Material from one of the plaques (clone #26) was streaked onto LB agar plates containing 30 μg/ml kanamycin (Km) to select carrier-state bacteria HB10Y(φ6-npt) bearing the recombinant virus. These could be stably propagated on Km-containing LB agar or in LB medium without loosing the npt gene, as judged by agarose gel electrophoresis of viral dsRNA and RT-PCR with npt-specific primers 5′-CAAGGAATTCCATGGGCCATATTCAACGGGAAA-3′ (SEQ ID NO:3) and 5′-CCAGGATCCTTTAAAAAAACTCATCGAGCATCAAATGAAACT-3′ (SEQ ID NO:4).

As expected, dsRNA segment M of the φ6-npt virus (M-npt), was longer than wild-type M, whereas φ6-npt L and S segments had regular lengths (FIG. 2A, lanes φ6 and K).

1.3. Constructing φ6-bla Carrier-state Cells

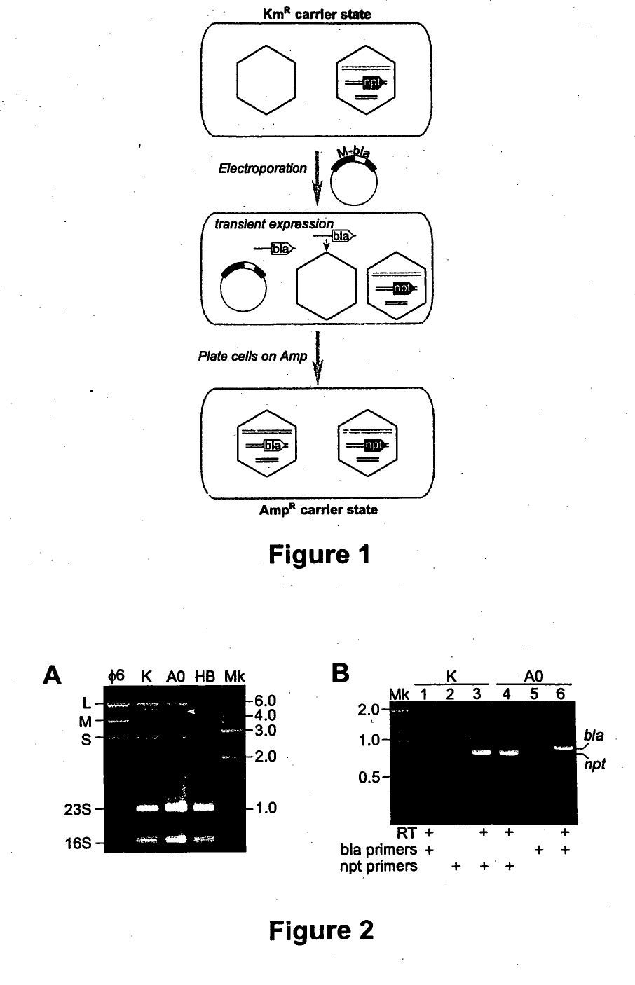

Constructing φ6-npt involved manipulations with purified RNAs and viral procapsids (PCs) in vitro, followed by spheroplast infection (Bamford et al., 1995). To avoid these technical difficulties when preparing φ6-bla virus, we used a plasmid-based strategy (FIG. 1) first developed by Mindich and colleagues (Mindich, 1999b). HB10Y(φ6-npt) cells were transformed with plasmid pEM38 that encodes the φ6 M segment containing the ampicillin resistance marker bla.

For the transformation, electrocompetent HB10Y(φ6-npt) cells were prepared as described (Lyra et al., 1991). These (40 μl) were electroporated with 0.1 mg/ml pEM38. The cell suspension was diluted with 1 ml of LB containing 1 mM MgSO₄, incubated at 28° C. for 2 h, and plated onto LB agar containing 150 μg/ml ampicillin.

pEM38 can not replicate in P. syringae but it can direct transient expression of the recombinant M segment, as previously shown for other E. coli plasmids (Mindich, 1999b). Some of the RNA transcripts can be packaged by PCs, present in the HB10Y(φ6-npt) cytoplasm, giving rise to 46-bla virus. Indeed, Amp-resistant colonies (10¹ to 10² μg⁻¹ DNA) appeared after 48-72 h of incubation at 28° C. on pEM38—but not on mock-transformed plates. One of the Amp-resistant clones, which could be stably propagated in the presence of Amp, was used for subsequent experiments. Electrophoretic analysis of the φ6-bla dsRNA genomic segments revealed the presence of two M segment species, M-npt and a new segment, M-bla, migrating between M-npt and wt M (FIG. 2A, lane A0).

1.4. Carrier State Bacteria Contain RNA-encoded Antibiotic Resistance Genes

We carried out RT-PCR analysis to ensure that the bla gene was indeed encoded by 46-bla rather than by host DNA. The bla PCR product was readily detectable when nucleic acid extracted from HB10Y(φ6-bla) was reverse-transcribed and amplified using bla-specific primers (FIG. 2B, lane 6). However, no product appeared in the control when the RT step was performed without reverse transcriptase (lane 5). This strongly suggests the RNA nature of the bla gene. Using npt-specific primers, we also observed that HB10Y(φ6-bla) bacteria retain detectable amounts of the npt gene (lane 4), consistent with the electrophoretic analysis of HB10Y(φ6-bla) RNA. As expected, HB10Y(φ6-npt) cells contained only an RNA-encoded npt gene (lanes 1-3).

### Example 2

### Directed Evolution of β-lactamase in φ6-bla Carrier-state Cells

2.1. P. syringae Carrying φ6—but not DNA-encoded bla Quickly Adapt to Cefotaxime

Wild-type TEM-1 β-lactamase encoded by φ6-bla hydrolyzes penicillin β-lactam antibiotics (e.g. Amp), but can not efficiently cleave third generation cefalosporins such as cefotaxime (Ctx). Since several Ctx-resistant β-lactamase variants have been reported (Bradford, 2001; Orencia et al., 2001), we investigated whether these could be selected using the carrier-state bacteria. HB10Y(φ6-bla) cells were plated onto LB agar containing either 150 μg/ml Amp or 50 μg/ml Ctx and incubated at 28° C. As a control, we used HB10Y cells transformed with a broad-range plasmid pLM254, whose bla gene is identical to that inserted into 46-bla (Mindich et al., 1985). Both HB10Y(φ6-bla) and HB10Y(pLM254) grew equally well on Amp medium (FIG. 3A). On Ctx medium, HB10Y(φ6-bla) formed slowly growing colonies of various sizes with an average frequency of ˜4 cfu (colony forming units) per 10⁶ cfu on Amp medium; no colonies were detected in the case of HB10Y(pLM254) by 96 h incubation (FIG. 3A). Because the abundance of pLM254 within cells is comparable to that of M-bla (not shown), we conclude that Ctx-resistant mutants appear considerably more often when bla is encoded by the M segment of φ6, rather than by plasmid DNA.

2.2. HB10Y(46-bla) Cells can Gradually Adapt to High Cefotaxime Concentrations

When the above experiment was repeated using ≧100 μg/ml Ctx, no growth was detected even on the plates with HB10Y(φ6-bla). We therefore tested the possibility that increased Ctx resistance can be developed by gradually increasing the concentration of Ctx and selecting the best growers. HB10Y(46-bla) cells were passed 10 times with the Ctx concentration being elevated from 10 to 4000 μg/ml as shown in FIG. 3B. The initial HB10Y(46-bla) stock was referred to as A0 and the cells obtained from different Ctx passages were called C1, C2, . . . , C10. On average, 10⁷-10⁸ Amp cfu were plated onto several petri dishes and the 20-40 largest colonies were picked and pooled after 48 h incubation. After a brief propagation (8-12 h, 28° C.) in LB medium containing Ctx at ¼ of the plate concentration, the cells were subjected to the next round of selection. Repeating this procedure several times it was possible to obtain P. syringae that were resistant to 2000-4000 μg/ml cefotaxime.

Several analyses were used to verify the presence of φ6-bla throughout the adaptation process. First, cellular RNA was studied by agarose gel-electrophoresis and RT-PCR using bla-specific primers (FIG. 3C). M segments of increased mobility were clearly present in all samples from C1 to C10, which correlated with the presence of the bla PCR fragment. M-bla was relatively sparse in C1 cells as judged by the reproducibly weak RT-PCR signal and the dominance of M-npt over M-bla on the RNA gel (lane C1). However, the amount of M-bla in C2 to C10 is notably higher than in A0. The M-npt band disappeared from the RNA pattern at C2.

In the second analysis, cellular proteins were separated by SDS-PAGE and subjected to immunoblotting with polyclonal antisera against φ6 proteins P1, P2, P4, and P8, components of φ6 nucleocapsids (FIG. 3D). Corresponding protein bands were detected in A0 and C1 to C10. The major φ6 capsid protein, P1, was also visible on Coomassie-stained gels.

Finally, when carrier-state bacteria were examined by electron microscopy, φ6 subviral particles and enveloped virions were observed in the cytopasm of A0, C1, C4, C7 and C10 cells, but not in the HB10Y control (FIG. 3E, and not shown).

### Example 3

### Analysis of the bla Evolution Results

3.1. Preparation of Total RNA from Carrier-state Bacteria

Bacterial cells pooled from 20-40 carrier-state colonies or pelleted from 1.5-ml liquid cultures were resuspended in 300 μl of 50 mM Tris-HCl, pH 8.0, 100 mM EDTA, 8% (v/w) sucrose. Lysozyme was added to 1 mg/ml and the mixture was incubated for 5 min at room temperature. Cells were lysed by 1% SDS for 3-5 min. SDS and most of the chromosomal DNA were precipitated by 1.5 M potassium acetate, pH 7.5 on ice. RNA was precipitated from the supernatant fraction by the addition of 0.7 volumes of isopropanol. The RNA pellet was dissolved in 400 μl TE (10 mM Tris-HCl, pH 8.0; 1 mM EDTA), extracted successively with equal volumes of phenol-chloroform and chloroform, and re-precipitated with ethanol. The pellet was washed with 70% ethanol and dissolved in 100 μl of sterile water.

3.2. RT-PCR and Cloning of the bla Gene

To obtain cDNA copies of the virus-encoded bla gene, total RNA (1 to 5 μg) from carrier-state bacteria was mixed with 10 pmol of the reverse transcription primer (5′-CTATCGAGCACAGCGCCAACT-3′) (SEQ ID NO:5), denatured by boiling for 1 min and chilled on ice. Reverse transcription was performed using AMV-RT (Sigma) at 45° C. for 1 h as recommended. The bla cDNA was PCR amplified using a mixture of Pfu and Taq DNA polymerases and the primers 5′-CCGAATTCATAAGGAAGCATATGAGTATTCA-3′ (SEQ ID NO:6 and 5′-CAACTTTTACGCTGGTGCTATACAACGACT-3′ (SEQ ID NO:7). HindIII-EcoRI cut PCR products were ligated with a similarly treated pSU 18 vector and transformed into E. coli DH5α. Cloned bla sequences were determined using a commercial automated sequencing facility (MWG-Biotech). Throughout the paper, amino acid numbering is according to (Ambler et al., 1991), which exceeds the physical number by 2.

3.3. Gene bla from Ctx-adapted Carrier State P. syringae Cells Confers Ctx Resistance in E. coli

To characterize the possible effect of cefotaxime selection on the β-lactamase gene, bla cDNA from A0, C1-C4, C7 and C10 passages was cloned into pSU18 (E. coli plasmid containing chloramphenicol (Cm) resistance marker) under control of the lac promoter. E. coli DH5α was transformed with the resulting plasmid libraries and plated onto Cm medium. Because existing cefotaxime-specific β-lactamases are also resistant to ampicillin (Bradford, 2001), we used plates with a low Amp concentration (50 μg/ml) to screen the libraries for clones containing the bla insert. A sufficient amount of β-lactamase was produced from the lac promoter without induction. Plasmids from the Amp-resistant clones (isolated from the master Cm plates) always contained the bla inserts. Conversely, several randomly selected clones that were resistant to Cm but not to Amp were the same size as the pSU18 vector.

We next examined whether E. coli containing pSU18 with bla inserts originating from φ6-bla are also resistant to Ctx. For this purpose, ˜10⁶ cells were transferred from colonies grown on Cm,—to plates containing 5 or 10 μg/ml Ctx. Of the 50-100 colonies analyzed for each library, 22% of the C1-derived bla clones were indeed resistant to 5 μg/ml Ctx. In the case of C2-, C3-, C4-, C7- and C10-derived libraries, the fraction of Ctx-resistant bla clones was 72, 81, 93, 100 and 100%, respectively, with most of the clones growing in the presence of 5 and 10 μg/ml Ctx. No Ctx-resistant colonies were detected in the A0-derived library.

3.4. Changes in bla Sequence During Adaptation to Cefotaxime

Complete bla sequences from several Ctx resistant clones were determined for each library (FIG. 4). Two bla alleles were found in the A0 library. One of these was the wild-type allele, occurring at an apparent frequency of 0.22, while the other one contained a single U→C mutation that changed F24 to S and occurred at an apparent frequency of 0.78. Surprisingly, multiple mutations were found in bla sequences from initial Ctx passages, one segment often containing several substitutions (up to 9 in C1; FIG. 5A). Most of the changes were transitions (FIG. 5B).

In addition to clone-specific mutations, two point mutations, F24S and a G→A substitution leading to the G238S mutation on the protein level, were detected in most bla sequences from C1 and subsequent passages. Beginning at C2, all sequences contained yet another common substitution, G→A, that changed E104 to K (compare C1 and C2 in FIG. 4). Interestingly, most clones in C4 and all clones from C7 and C10 contained only F24S, E104K and G238S mutations, with no other mutations being detected (FIG. 4).

To ensure that the accumulation of bla mutants after the antibiotic change was a specific effect of Ctx, we carried out a mock selection experiment. A0 cells were plated onto dishes containing 150 mg/ml Amp and incubated for 48 h at 28° C. (passage A1). dsRNA purified from 40 pooled colonies was used to construct an RT-PCR library in E. coli as described above. No Ctx-resistant clones were found and no other alleles were detected besides wt and F24S (with frequencies of 0.4 and 0.6, respectively).

Since 78% of the Amp-resistant clones from the C1 library failed to grow in the presence of Ctx, we determined bla sequences from seven Ctx-sensitive clones. All sequences contained one or several mutations on the wt or F24S background, the overall picture being similar to Ctx-resistant clones (not shown). The only difference was that none of the Ctx-sensitive clones contained the G238S substitution. We conclude that the E104K and G238S mutations were critical to enable Ctx hydrolysis. Indeed, both mutations map to the enzyme active site and are often observed in Ctx-resistant bacteria (Bradford, 2001; Orencia et al., 2001).

The overall dynamics of the bla population adapting to Ctx is apparent from the percent identity plots (FIG. 5C). A relatively homogenous population in A0 (and A1) was diversified dramatically in C1 and C2. After the appropriate mutations were accumulated, the population regained homogeneity in C4-C7. Further passages did not change the genetic structure of the population. Importantly, the genetic heterogeneity in C2 and C3 was clearly higher than in A0, and the M-bla segment was more abundant in C2 and C3 than in A0 (FIG. 3C). Therefore, possible effects of RT-PCR derived mutations can be excluded.

### REFERENCES

Altamirano, M. M., Blackburn, J. M., Aguayo, C. and Fersht, A. R. (2000) Directed evolution of new catalytic activity using the alpha/beta-barrel scaffold. Nature, 403, 617-22.

Ambler, R. P., Coulson, A. F., Frere, J. M., Ghuysen, J. M., Joris, B., Forsman, M., Levesque, R. C., Tiraby, G. and Waley, S. G. (1991) A standard numbering scheme for the class A beta-lactamases. Biochem J. 276, 269-70.

Amstutz, P., Forrer, P., Zahnd, C. and Pluckthun, A. (2001) In vitro display technologies: novel developments and applications. Curr Opin Biotechnol, 12, 400-5.

Arnold, F. H. (2001) Combinatorial and computational challenges for biocatalyst design. Nature, 409, 253-7.

Bamford, D. H. and Mindich, L. (1980) Electron microscopy of cells infected with nonsense mutants of bacteriophage phi 6. Virology, 107, 222-8.

Bamford, D. H., Ojala, P. M., Frilander, M., Walin, L. and Bamford, J. K. H. (1995) Isolation, purification, and function of assembly intermediates and subviral particles of bacteriophages RPD1 and phi6. In Adolph, K. W. (ed.) Methods in molecular genetics. Academic Press, San Diego, Vol. 6, pp. 455-74.

Barstead, R. (2001) Genome-wide RNAi. Curr Opin Chem Biol, 5, 63-6.

Bartolome, B., Jubete, Y., Martinez, E. and de la Cruz, F. (1991) Construction and properties of a family of pACYC184-derived cloning vectors compatible with pBR322 and its derivatives. Gene, 102, 75-8.

Baulcombe, D. (2002) RNA silencing. Curr Biol, 12, R82-4.

Bittker, J. A., Le, B. V. and Liu, D. R. (2002a) Nucleic acid evolution and minimization by nonhomologous random recombination. Nat Biotechnol, 20, 1024-9.

Bittker, J. A., Phillips, K. J. and Liu, D. R. (2002b) Recent advances in the in vitro evolution of nucleic acids. Curr Opin Chem Biol, 6, 367-74.

Bomscheuer, U. T. and Pohl, M. (2001) Improved biocatalysts by directed evolution and rational protein design. Curr Opin Chem Biol, 5, 137-43.

Bradford, P. A. (2001) Extended-spectrum beta-lactamases in the 21st century: characterization, epidemiology, and detection of this important resistance threat. Clin Microbiol Rev, 14, 933-51, table of contents.

Brown, D. and Gold, L. (1995) Selection and characterization of RNAs replicated by Q beta replicase. Biochemistry, 34, 14775-82.

Buck, K. W. (1996) Comparison of the replication of positive-stranded RNA viruses of plants and animals. Adv Virus Res, 47, 159-251.

Burgstaller, P., Jenne, A. and Blind, M. (2002) Aptamers and aptazymes: accelerating small molecule drug discovery. Curr Opin Drug Discov Devel, 5, 690-700.

Carter, P. (2001) Improving the efficacy of antibody-based cancer therapies. Nat Rev Cancer, 1, 118-29.

Chao, L., Rang, C. U. and Wong, L. E. (2002) Distribution of spontaneous mutants and inferences about the replication mode of the RNA bacteriophage phi6. J Virol, 76, 3276-81.

Chen, W. and Georgiou, G. (2002) Cell-Surface display of heterologous proteins: From high-throughput screening to environmental applications. Biotechnol Bioeng, 79, 496-503.

Cobaleda, C. and Sanchez-Garcia, I. (2001) RNase P: from biological function to biotechnological applications. Trends Biotechnol, 19, 406-11.

Coco, W. M., Levinson, W. E., Crist, M. J., Hektor, H. J., Darzins, A., Pienkos, P. T., Squires, C. H. and Monticello, D. J. (2001) DNA shuffling method for generating highly recombined genes and evolved enzymes. Nat Biotechnol, 19, 354-9.

Cogoni, C. (2001) Homology-dependent gene silencing mechanisms in fungi. Annu Rev Microbiol, 55, 381-406.

Cohen, N., Abramov, S., Dror, Y. and Freeman, A. (2001) In vitro enzyme evolution: the screening challenge of isolating the one in a million. Trends Biotechnol, 19, 507-10.

de Feyter, R. and Li, P. (2000) Technology evaluation: HIV ribozyme gene therapy, Gene Shears Pty Ltd. Curr Opin Mol Ther, 2, 332-5.

Domingo, E., Biebricher, C. K., Eigen, M. and Holland, J. J. (2001) Quasispecies and RNA virus evolution: principles and consequences. Landes Bioscience, Georgetown, Tex.

Domingo, E., Escannis, C., Sevilla, N., Moya, A., Elena, S. F., Quer, J., Novella, I. S. and Holland, J. J. (1996) Basic concepts in RNA virus evolution. Faseb J. 10, 859-64.

Domingo, E., Menendez-Arias, L., Quinones-Mateu, M. E., Holguin, A., Gutierrez-Rivas, M., Martinez, M. A., Quer, J., Novella, I. S. and Holland, J. J. (1997) Viral quasispecies and the problem of vaccine-escape and drug-resistant mutants. Prog Drug Res, 48, 99-128.

Doudna, J. A. and Cech, T. R. (2002) The chemical repertoire of natural ribozymes. Nature, 418, 222-8.

Drake, J. W. and Holland, J. J. (1999) Mutation rates among RNA viruses. Proc Natl Acad Sci USA, 96, 13910-3.

Eigen, M. (1996) On the nature of virus quasispecies. Trends Microbiol, 4, 216-8.

Farci, P., Shimoda, A., Coiana, A., Diaz, G., Peddis, G., Melpolder, J. C., Strazzera, A., Chien, D. Y., Munoz, S. J., Balestrieri, A., Purcell, R. H. and Alter, H. J. (2000) The outcome of acute hepatitis C predicted by the evolution of the viral quasispecies. Science, 288, 339-44.

Farinas, E. T., Bulter, T. and Arnold, F. H. (2001) Directed enzyme evolution. Curr Opin Biotechnol, 12, 545-51.

Forrer, P., Jung, S. and Pluckthun, A. (1999) Beyond binding: using phage display to select for structure, folding and enzymatic activity in proteins. Curr Opin Struct Biol, 9, 514-20.

Fouchier, R. A., Kuiken, T., Schutten, M., Van Amerongen, G., Van Doomum, G. J., Van Den Hoogen, B. G., Peiris, M., Lim, W., Stohr, K. and Osterhaus, A. D. (2003) Aetiology: Koch's postulates fulfilled for SARS virus. Nature, 423, 240.

Gallie, D. R. and Walbot, V. (1990) RNA pseudoknot domain of tobacco mosaic virus can functionally substitute for a poly(A) tail in plant and animal cells. Genes Dev, 4, 1149-57.

Gibbs, M. J., Koga, R., Moriyama, H., Pfeiffer, P. and Fukuhara, T. (2000) Phylogenetic analysis of some large double-stranded RNA replicons from plants suggests they evolved from a defective single-stranded RNA virus. J Gen Virol, 81, 227-33.

Griffiths, A. D. and Tawfik, D. S. (2003) Directed evolution of an extremely fast phosphotriesterase by in vitro compartmentalization. Embo J, 22, 24-35.

Grosshans, H. and Slack, F. J. (2002) Micro-RNAs: small is plentiful. J Cell Biol, 156, 17-21.

Hannon, G. J. (2002) RNA interference. Nature, 418, 244-51.

Harrigan, P. R. and Alexander, C. S. (1999) Selection of drug-resistant HIV. Trends Microbiol, 7, 120-3.

Hoogstraten, D., Qiao, X., Sun, Y., Hu, A., Onodera, S. and Mindich, L. (2000) Characterization of phi8, a bacteriophage containing three double-stranded RNA genomic segments and distantly related to Phi6. Virology, 272, 218-24.

Hudson, P. J. and Souriau, C. (2003) Engineered antibodies. Nat Med, 9, 129-34.

Jacque, J. M., Triques, K. and Stevenson, M. (2002) Modulation of HIV-1 replication by RNA interference. Nature, 418, 435-8.

Jaschke, A. (2001) Artificial ribozymes and deoxyribozymes. Curr Opin Struct Biol, 11, 321-6.

Jeruzalmi, D. and Steitz, T. A. (1998) Structure of T7 RNA polymerase complexed to the transcriptional inhibitor T7 lysozyme. Embo J, 17, 4101-13.

Kamath, R. S., Fraser, A. G., Dong, Y., Poulin, G., Durbin, R., Gotta, M., Kanapin, A., Le Bot, N., Moreno, S., Sohrmann, M., Welchman, D. P., Zipperlen, P. and Ahringer, J. (2003) Systematic functional analysis of the Caenorhabditis elegans genome using RNAi. Nature, 421, 231-7.

Kolkman, J. A. and Stemmer, W. P. (2001) Directed evolution of proteins by exon shuffling. Nat Biotechnol, 19, 423-8.

Kurtzman, A. L., Govindarajan, S., Vahle, K., Jones, J. T., Heinrichs, V. and Patten, P. A. (2001) Advances in directed protein evolution by recursive genetic recombination: applications to therapeutic proteins. Curr Opin Biotechnol, 12, 361-70.

Lai, M. M. (1995) The molecular biology of hepatitis delta virus. Annu Rev Biochem, 64, 259-86.

Leathers, V., Tanguay, R., Kobayashi, M. and Gallie, D. R. (1993) A phylogenetically conserved sequence within viral 3′ untranslated RNA pseudoknots regulates translation. Mol Cell Biol, 13, 5331-47.

Lemon, B. and Tjian, R. (2000) Orchestrated response: a symphony of transcription factors for gene control. Genes Dev, 14, 2551-69.

Lindbo, J. A., Fitzmaurice, W. P. and della-Cioppa, G. (2001) Virus-mediated reprogramming of gene expression in plants. Curr Opin Plant Biol, 4, 181-5.

Long-McGie, J., Liu, A. D. and Schellenberger, V. (2000) Rapid in vivo evolution of a beta-lactamase using phagemids. Biotechnol Bioeng, 68, 121-5.

Lum, L., Yao, S., Mozer, B., Rovescalli, A., Von Kessler, D., Nirenberg, M. and Beachy, P. A. (2003) Identification of Hedgehog pathway components by RNAi in Drosophila cultured cells. Science, 299, 2039-45.

Lyra, C., Savilahti, H. and Bamford, D. H. (1991) High-frequency transfer of linear DNA containing 5′-covalently linked terminal proteins: electroporation of bacteriophage RPD1 genome into Escherichia coli. Mol Gen Genet, 228, 65-9.

Marra, M. A., Jones, S. J., Astell, C. R., Holt, R. A., Brooks-Wilson, A., Butterfield, Y. S., Khattra, J., Asano, J. K., Barber, S. A., Chan, S. Y., Cloutier, A., Coughlin, S. M., Freeman, D., Girn, N., Griffith, O. L., Leach, S. R., Mayo, M., McDonald, H., Montgomery, S. B., Pandoh, P. K., Petrescu, A. S., Robertson, A. G., Schein, J. E., Siddiqui, A., Smailus, D. E., Stott, J. M., Yang, G. S., Plummer, F., Andonov, A., Artsob, H., Bastien, N., Bernard, K., Booth, T. F., Bowness, D., Drebot, M., Fernando, L., Flick, R., Garbutt, M., Gray, M., Grolla, A., Jones, S., Feldmann, H., Meyers, A., Kabani, A., Li, Y., Normand, S., Stroher, U., Tipples, G. A., Tyler, S., Vogrig, R., Ward, D., Watson, B., Brunharn, R. C., Krajden, M., Petric, M., Skowronski, D. M., Upton, C. and Roper, R. L. (2003) The Genome Sequence of the SARS-Associated Coronavirus. Science, 1, 1.

McCaffrey, A. P., Nakai, H., Pandey, K., Huang, Z., Salazar, F. H., Xu, H., Wieland, S. F., Marion, P. L. and Kay, M. A. (2003) Inhibition of hepatitis B virus in mice by RNA interference. Nat Biotechnol, 12, 12.

McManus, M. T. and Sharp, P. A. (2002) Gene silencing in mammals by small interfering RNAs. Nat Rev Genet, 3, 737-47.

Miller, W. A. and Koev, G. (1998) Getting a handle on RNA virus recombination. Trends Microbiol, 6, 421-3.

Mindich, L. (1988) Bacteriophage phi 6: a unique virus having a lipid-containing membrane and a genome composed of three dsRNA segments. Adv Virus Res, 35, 137-76.

Mindich, L. (1999a) Precise packaging of the three genomic segments of the double-stranded-RNA bacteriophage phi6. Microbiol Mol Biol Rev, 63, 149-60.

Mindich, L. (1999b) Reverse genetics of dsRNA bacteriophage phi 6. Adv Virus Res, 53, 341-53.

Mindich, L., MacKenzie, G., Strassman, J., McGraw, T., Metzger, S., Romantschuk, M. and Bamford, D. (1985) cDNA cloning of portions of the bacteriophage phi 6 genome. J Bacteriol, 162, 992-9.

Mindich, L., Qiao, X., Qiao, J., Onodera, S., Romantschuk, M. and Hoogstraten, D. (1999) Isolation of additional bacteriophages with genomes of segmented double-stranded RNA. J Bacteriol, 181, 4505-8.

Myers, J. W., Jones, J. T., Meyer, T. and Ferrell, J. E. (2003) Recombinant Dicer efficiently converts large dsRNAs into siRNAs suitable for gene silencing. Nat Biotechnol, 21, 324-8.

Negroni, M. and Buc, H. (2001) Mechanisms of retroviral recombination. Annu Rev Genet, 35, 275-302.

Nichol, S. T., Arikawa, J. and Kawaoka, Y. (2000) Emerging viral diseases. Proc Natl Acad Sci USA, 97, 12411-2.

Nixon, A. E., Firestine, S. M., Salinas, F. G. and Benkovic, S. J. (1999) Rational design of a scytalone dehydratase-like enzyme using a structurally homologous protein scaffold. Proc Natl Acad Sci USA, 96, 3568-71.

Novina, C. D., Murray, M. F., Dykxhoorn, D. M., Beresford, P. J., Riess, J., Lee, S. K., Collman, R. G., Lieberman, J., Shankar, P. and Sharp, P. A. (2002) siRNA-directed inhibition of HIV-1 infection. Nat Med, 8, 681-6.

Olkkonen, V. M. and Bamford, D. H. (1989) Quantitation of the adsorption and penetration stages of bacteriophage phi 6 infection. Virology, 171, 229-38.

Olkkonen, V. M., Gottlieb, P., Strassman, J., Qiao, X. Y., Bamford, D. H. and Mindich, L. (1990) In vitro assembly of infectious nucleocapsids of bacteriophage phi 6: formation of a recombinant double-stranded RNA virus. Proc Natl Acad Sci U S A, 87, 9173-7.

Olsthoom, R. C., Mertens, S., Brederode, F. T. and Bol, J. F. (1999) A conformational switch at the 3′ end of a plant virus RNA regulates viral replication. Embo J, 18, 4856-64.

O'Neil, K. T. and Hoess, R. H. (1995) Phage display: protein engineering by directed evolution. Curr Opin Struct Biol, 5, 443-9.

Onodera, S., Olkkonen, V. M., Gottlieb, P., Strassman, J., Qiao, X. Y., Bamford, D. H. and Mindich, L. (1992) Construction of a transducing virus from double-stranded RNA bacteriophage phi6: establishment of carrier states in host cells. J Virol, 66, 190-6.

Onodera, S., Qiao, X., Gottlieb, P., Strassman, J., Frilander, M. and Mindich, L. (1993) RNA structure and heterologous recombination in the double-stranded RNA bacteriophage phi 6. J Virol, 67, 4914-22.

Onodera, S., Qiao, X., Qiao, J. and Mindich, L. (1995) Acquisition of a fourth genomic segment in bacteriophage phi 6, a bacteriophage with a genome of three segments of dsRNA. Virology, 212, 204-12.

Onodera, S., Qiao, X., Qiao, J. and Mindich, L. (1998) Directed changes in the number of double-stranded RNA genomic segments in bacteriophage phi6. Proc Natl Acad Sci USA, 95, 3920-4.

Onodera, S., Sun, Y. and Mindich, L. (2001) Reverse genetics and recombination in Phi8, a dsRNA bacteriophage. Virology, 286, 113-8.

Orencia, M. C., Yoon, J. S., Ness, J. E., Stemmer, W. P. and Stevens, R. C. (2001) Predicting the emergence of antibiotic resistance by directed evolution and structural analysis. Nat Struct Biol, 8, 238-42.

Palese, P. (1998) RNA virus vectors: where are we and where do we need to go? Proc Natl Acad Sci USA, 95, 12750-2.

Pasloske, B. L. (2001) Ribonuclease inhibitors. Methods Mol Biol, 160, 105-11.

Pekarik, V., Bourikas, D., Miglino, N., Joset, P., Preiswerk, S. and Stoeckli, E. T. (2003) Screening for gene function in chicken embryo using RNAi and electroporation. Nat Biotechnol, 21, 93-6.

Petrounia, I. P. and Arnold, F. H. (2000) Designed evolution of enzymatic properties. Curr Opin Biotechnol, 11, 325-30.

Pohorille, A. and Deamer, D. (2002) Artificial cells: prospects for biotechnology. Trends Biotechnol, 20, 123-8.

Poranen, M. M., Paatero, A. O., Tuma, R. and Bamford, D. H. (2001) Self-assembly of a viral molecular machine from purified protein and RNA constituents. Mol Cell, 7, 845-54.

Qiao, X., Qiao, J. and Mindich, L. (1997) An in vitro system for the investigation of heterologous RNA recombination. Virology, 227, 103-10.

Qiao, X., Qiao, J., Onodera, S. and Mindich, L. (2000) Characterization of phi 13, a bacteriophage related to phi 6 and containing three dsRNA genomic segments. Virology, 275, 218-24.

Quemeneur, E., Moutiez, M., Charbonnier, J. B. and Menez, A. (1998) Engineering cyclophilin into a proline-specific endopeptidase. Nature, 391, 301-4.

Rader, C. and Barbas, C. F., 3rd. (1997) Phage display of combinatorial antibody libraries. Curr Opin Biotechnol, 8, 503-8.

Robbins, P. D., Tahara, H. and Ghivizzani, S. C. (1998) Viral vectors for gene therapy. Trends Biotechnol, 16, 35-40.

Robertson, M. P. and Ellington, A. D. (2001) In vitro selection of nucleoprotein enzymes. Nat Biotechnol, 19, 650-5.

Sachs, A. B. (2000) Cell cycle-dependent translation initiation: IRES elements prevail. Cell, 101, 243-5.

Sachs, A. B. and Buratowski, S. (1997) Common themes in translational and transcriptional regulation. Trends Biochem Sci, 22, 189-92.

Schatz, P. J., Cull, M. G., Martin, E. L. and Gates, C. M. (1996) Screening of peptide libraries linked to lac repressor. Methods Enzymol, 267, 171-91.

Selifonova, O., Valle, F. and Schellenberger, V. (2001) Rapid evolution of novel traits in microorganisms. Appl Environ Microbiol, 67, 3645-9.

Soumillion, P. and Fastrez, J. U.-h.w.s.c.s.a.B.V.-K.C.-D.b.f.e.e.f.b.c.c.a.f. (2001) Novel concepts for selection of catalytic activity. Current Opinion in Biotechnology, 12, 387-394.

Stemmer, W. P. (1994a) DNA shuffling by random fragmentation and reassembly: in vitro recombination for molecular evolution. Proc Natl Acad Sci USA, 91, 10747-51.

Stemmer, W. P. (1994b) Rapid evolution of a protein in vitro by DNA shuffling. Nature, 370, 389-91.

Tawfik, D. S. and Griffiths, A. D. (1998) Man-made cell-like compartments for molecular evolution. Nat Biotechnol, 16, 652-6.

Theil, E. C. (1993) The IRE (iron regulatory element) family: structures which regulate mRNA translation or stability. Biofactors, 4, 87-93.

Trower, M. K. (1996) In vtro mutagenesis protocols. Humana Press, Totowa, N.J.

Vagner, S., Galy, B. and Pyronnet, S. (2001) Irresistible IRES. Attracting the translation machinery to internal ribosome entry sites. EMBO Rep, 2, 893-8.

van Roessel, P. and Brand, A. H. (2002) Imaging into the future: visualizing gene expression and protein interactions with fluorescent proteins. Nat Cell Biol, 4, E15-20.

Vance, V. and Vaucheret, H. (2001) RNA silencing in plants—defense and counterdefense. Science, 292, 2277-80.

White, L. K., Wright, W. E. and Shay, J. W. (2001) Telomerase inhibitors. Trends Biotechnol, 19, 114-20.

Wickner, R. B. (1996) Prions and RNA viruses of Saccharomyces cerevisiae. Annu Rev Genet, 30, 109-39.

Wilson, D. S. and Szostak, J. W. (1999) In vitro selection of functional nucleic acids. Annu Rev Biochem, 68, 611-47.

Winter, G. and Harris, W. J. (1993) Humanized antibodies. Immunol Today, 14, 243-6.

Wittrup, K. D. U.-h.w.s.c.s.a.B.V.-K.C.-F.c.c.f.c.a. (2001) Protein engineering by cell-surface display. Current Opinion in Biotechnology, 12, 395-399.

Yang, D., Buchholz, F., Huang, Z., Goga, A., Chen, C. Y., Brodsky, F. M. and Bishop, J. M. (2002) Short RNA duplexes produced by hydrolysis with Escherichia coli RNase III mediate effective RNA interference in mammalian cells. Proc Natl Acad Sci U S A, 99, 9942-7.

Zeenko, V. V., Ryabova, L. A., Spirin, A. S., Rothnie, H. M., Hess, D., Browning, K. S. and Hohn, T. (2002) Eukaryotic elongation factor 1A interacts with the upstream pseudoknot domain in the 3′ untranslated region of tobacco mosaic virus RNA. J Virol, 76, 5678-91.

Zhao, H., Chockalingam, K. and Chen, Z. (2002) Directed evolution of enzymes and pathways for industrial biocatalysis. Curr Opin Biotechnol, 13, 104-10.

1. A method for changing a target nucleic acid sequence, the method comprising:
a) providing nucleic acid target in a form that can be replicated by a polymerase devoid of the proof-reading function;
b) incorporating the nucleic acid target into the genome of an RNA virus or other RNA replicon where said nucleic acid target is replicated by the polymerase encoded by the RNA virus or other RNA replicon under conditions sufficient for template-directed nucleic acid synthesis in a living cell; and
c) recovering nucleic acid synthesis products, whose nucleotide sequence differs from the initial target sequence by at least one nucleotide.

2. The method according to claim 1, wherein said nucleic acid target encodes a polypeptide.

3. The method according to claim 1, wherein said polymerase is an RNA-dependent RNA polymerase.

4. The method according to claim 1, wherein said polymerase is an RNA-dependent DNA polymerase.

5. The method according to claim 1, wherein the nucleic acid synthesis products are recovered after selecting and/or screening nucleic acid synthesis products based on their properties.

6. The method according to claim 1, wherein said nucleic acid synthesis products are recovered after one or several rounds of selection and/or screening.

7. The method according to claim 1, wherein the method is specifically used for changing properties of proteins or nucleic acids in a desired manner.

8. The method according to claim 1, wherein the polymerase is a genetically modified or wild-type polymerase.

9. The method according to claim 1, wherein the RNA virus or other RNA replicon is genetically modified or wild-type.

10. The method according to claim 1, wherein the nucleic acid target is operably linked with determinants essential for detectable replication by the polymerase.

11. The method according to claim 1, wherein the RNA replicon is an RNA virus-like particle, viroid or RNA-based autonomous genetic element.

12. The method according to claim 1, wherein the nucleic acid encoding the polymerase and the target nucleic acid are distinct nucleic acids.

13. The method according to claim 1, wherein the nucleic acid target is a nucleic acid having detectable biological activity, preferably selected from the group comprising enzymatic, regulatory and specific binding activity.

14. The method according to claim 1, wherein the nucleic acid target encodes a protein having detectable biological activity, preferably selected from the group comprising enzymatic, regulatory and specific binding activity.

15. The method according to claim 1, wherein the nucleic acid target is RNA.

16. The method according to claim 1, wherein the nucleic acid target is DNA.

17. The method according to claim 1, wherein the nucleic acid synthesis products are RNA molecules.

18. The method according to claim 1, wherein the nucleic acid synthesis products are DNA molecules.

19. The method according to claim 1, wherein the RNA virus is an RNA bacteriophage.

20. The method according to claim 19, wherein the RNA virus is from a member of the Cystoviridae family, preferably from a bacteriophage selected from the group comprising φ6, φ7, φ8, φ9, φ10, φ11, φ12, φ13 and φ14, most preferably from bacteriophage φ6.

21. The method according to claim 1, wherein the replicable form of the nucleic acid target is replicated in a prokaryotic cell, preferably in a gram-negative bacterial cell, more preferably in a bacterial cell selected from the group comprising Pseudomonas sp., Escherichia sp. and Salmonella sp., most preferably in a cell of Pseudomonas syringae.

22. The method according to claim 1, wherein the replicable form of the nucleic acid target is replicated in a eukaryotic cell, such as mammalian, insect, plant or yeast cell.

23. The method according to claim 1, wherein the nucleic acid target is delivered into the living cell by using a suicide vector, preferably a DNA vector, most preferably a DNA plasmid.

24. The method according to claim 1, wherein a suicide vector, comprising a target nucleic acid operably linked with sequences sufficient for detectable replication by the viral replication apparatus, is used to incorporate said nucleic acid target into the genome of said RNA virus.

25. A system for changing a target nucleic acid sequence, which comprises
a target nucleic acid sequence operably linked with determinants essential for replication by an RNA synthesis apparatus of an RNA virus or another RNA replicon;
a living cell capable of supporting the replication of the RNA virus or other RNA replicon; and
a selection/screening procedure for selecting/screening a change in the properties of the nucleic acid synthesis products.

26. The system according to claim 25, wherein the RNA-synthesis apparatus is from a member of Cystoviridae family.

27. The system according to claim 25, wherein the living cells are bacteria, preferably gram-negative bacteria, more preferably bacteria selected from the group comprising Pseudomonas sp., Escherichia sp. and Salmonella sp., most preferably Pseudomonas syringae.

28. The system according to claim 25, wherein the cells are carrier-state cells or can be transformed into carrier state.

29. A kit for changing nucleic acid or protein sequences, which comprises:
a) a vector for transient expression of target nucleic acid in preselected cells that either are carrier-state or can be transformed into carrier state and/or
b) a genetically modified virus into where the target nucleic acid can be introduced; and/or
c) cells that either are carrier-state or can be transformed into carrier state.
